(19) United States
(12) Patent Application Publication
(10) Pub. No.: US 2016/0144184 A1
(43) Pub. Date: May 26, 2016

(54) Implantable Stimulator Device Having Charge Measuring Circuitry Useable in Conjunction with DC-Blocking Capacitors

(71) Applicant: Boston Scientific Neuromodulation Corporation, Valencia, CA (US)

(72) Inventor: Goran N. Marnfeldt, Valencia, CA (US)

(21) Appl. No.: 14/879,781

(22) Filed: Oct. 9, 2015

Related U.S. Application Data
(60) Provisional application No. 62/083,080, filed on Nov. 21, 2014.

(51) Int. Cl. A61N 1/36

(52) U.S. Cl. CPC A61N 1/36125; A61N 1/36128

(57) ABSTRACT

Improved circuitry for an Implantable Pulse Generator (IPG) is disclosed that allows much smaller-value DC-blocking capacitors to be used with supported electrodes—with capacitance values orders of magnitude smaller than those used in traditional IPGs. Such improved circuitry operates by alternating the direction of the current through the DC-blocking capacitor during the provision of a therapeutic current pulse. Such smaller-value DC-blocking capacitors do not take up significant space in the IPG, or surface area on the IPG's PCB. Additionally, the improved circuitry includes the ability to measure the current amplitude provided to selected electrodes—for example, to ensure that the sources are actually providing a prescribed current amplitude to the patient's tissue—and to provide for perfect active charge recovery.

### CROSS-REFERENCE TO RELATED APPLICATIONS

This is a non-provisional application of U.S. Provisional Patent Application Ser. No. 62/083,080, filed Nov. 21, 2014, which is incorporated herein by reference, and to which priority is claimed.

### FIELD OF THE INVENTION

The present invention relates to implantable stimulator devices, and in particular to minimizing the capacitance value of DC-blocking capacitors coupled in series with the electrodes in such devices.

### BACKGROUND

Implantable stimulation devices deliver electrical stimuli to nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Spinal Cord Stimulation (SCS) system, such as that disclosed in U.S. Pat. No. 6,516,227. However, the present invention may find applicability with any implantable medical device or in any implantable medical device system.

An SCS system typically includes an Implantable Pulse Generator (IPG) 10 shown in plan and cross-sectional views in FIGS. 1A and 1B. The IPG 10 includes a biocompatible device case 30 that holds the circuitry and battery 36 necessary for the IPG to function. The IPG 10 is coupled to electrodes 16 via one or more electrode leads 14 that form an electrode array 12. The electrodes 16 are configured to contact a patient's tissue and are carried on a flexible body 18, which also houses the individual lead wires 20 coupled to each electrode 16. The lead wires 20 are also coupled to proximal contacts 22, which are insertable into lead connectors 24 fixed in a header 28 on the IPG 10, which header can comprise an epoxy for example. Once inserted, the proximal contacts 22 connect to header contacts 26, which are in turn coupled by feedthrough pins 34 through a case feedthrough 32 to circuitry within the case 30.

In the illustrated IPG 10, there are thirty-two lead electrodes (E1-E32) split between four leads 14, with the header 28 containing a 2×2 array of lead connectors 24. However, the number of leads and electrodes in an IPG is application specific and therefore can vary. In a SCS application, the electrode leads 14 are typically implanted proximate to the dura in a patient's spinal cord, and when a four-lead IPG 10 is used, these leads are usually split with two on each of the right and left sides of the dura. The proximal electrodes 22 are tunneled through the patient's tissue to a distant location such as the buttocks where the IPG case 30 is implanted, at which point they are coupled to the lead connectors 24. A four-lead IPG 10 can also be used for Deep Brain Stimulation (DBS) in another example. In other IPG examples designed for implantation directly at a site requiring stimulation, the IPG can be lead-less, having electrodes 16 instead carried by the case of the IPG for contacting the patient's tissue.

As shown in the cross section of FIG. 1B, the IPG 10 includes a printed circuit board (PCB) 40. Electrically coupled to the PCB 40 are the battery 36, which in this example is rechargeable; other circuitry 50a and 50b coupled to top and bottom surfaces of the PCB; a communication coil 42 for wirelessly communicating with an external controller (not shown); a charging coil 44 for wirelessly receiving a magnetic charging field from an external charger (not shown) for recharging the battery 36; and the feedthrough pins 34 (connection not shown). If battery 36 is permanent and not rechargeable, charging coil 44 would be unnecessary. (Further details concerning operation of the coils 42 and 44 and the external devices with which they communicate can be found in U.S. Patent Application Publication 2015/0080982).

FIGS. 2A and 2B show some of the other circuitry 50a or 50b present in the IPG 10, and in particular FIG. 2A shows current distribution circuitry used to establish a current at any of the lead electrodes 16, which currents may comprise monophasic or multi-phasic current pulses. In this example, the current distribution circuitry includes a number of current sources (PDACs 52) and current sinks (NDACs 54). The PDACs 52 and NDACs 54 comprise Digital-to-Analog converters (DACs) able to respectively source and sink current pulses Iout, the shape of which may be controllable in accordance with digital control signals reflecting various parameters of pulse Iout, such as amplitude, pulse width, frequency, and polarity, as described in detail later. (“PDAC” and “NDAC” are so called because they are typically made from P-channel and N-channel transistors respectively).

The sourced or sunk current from one or more active PDACs 52 or NDACs 54 is directed to selected electrodes 16 via switch matrices 56P and 56N, which are also digitally controlled (not shown). Note that the current distribution circuitry in this example also supports selection of the conductive case 30 as an electrode (Ecase 30), which case electrode 30 is typically selected for monopolar stimulation, as is well known. DACs 52 and 54 can also comprise voltage sources. The sourcing components (PDACs 52, matrix 56P) and the sinking components (NDACs 54, matrix 56N) can individually also be considered as current distribution circuitry.

Proper control of the DACs 52 and 54 and the switching matrices 56 allows any of the electrodes 16 to act as anodes or cathodes to create a current through a patient's tissue, R, hopefully with good therapeutic effect. In the example shown, PDAC 52₂ is controlled to source a current pulse Iout to anode electrode E1 via switch matrix 56P, while NDAC 54₁ is controlled to sink that pulse from cathode electrode E2 via switching matrix 56N. Power for the current distribution circuitry is provided by a compliance power supply voltage V+, as described in further detail in U.S. Patent Application Publication 2013/0289665 for example. More than one anode electrode and more than one cathode electrode may be selected at one time, and thus current can flow between two or more of the electrodes 16.

Other current distribution circuitries can also be used in IPG 10. In an example not using switching matrices 56, each electrode node Ei′ (explained further below) can be provided with a digitally-controllable PDAC 52i and NDAC 54i dedicated to that electrode node Ei′, such as is disclosed in U.S. Pat. No. 6,181,969 for example. In another example, the PDACs 52 and NDACs 54 may provide currents of fixed amplitudes, with multiple of these DACs being selected by the switching matrices 56 to provide a sum of their currents at a selected electrode, such as described in U.S. Patent Application Publications 2007/0038250 and 2007/0100399.

Much of the current distribution circuitry of FIG. 2A, including the DACs 52 and 54 and the switch matrices 56, can be integrated on an Application Specific Integrated Circuit (ASIC) 60, as shown in FIG. 2B. In the example shown, ASIC 60 contains circuitry to support sixteen lead electrodes 16 and the case 30 electrode. In a 32-electrode IPG 10, two such ASIC 60a and 60b are used, with the case electrode Ecase being activated in only one of the ASICs 60a or 60b, in effect creating a 33-electrode device. ASICs 60a and 60b can be identically fabricated, and controlled by a microcontroller integrated circuit (μC) 62 via a digital bus 61, as disclosed in U.S. Patent Application Publications 2012/0095529, 2012/0092031, and 2012/0095519. ASICs 60a and 60b may also contain other circuitry useful in the IPG 10, such as battery charging and protection circuitry (for interfacing off chip with the battery 36 and charging coil 44), telemetry circuitry (for interfacing off chip with telemetry coil 42), various measurement circuits, etc. Of course, only one ASIC 60 may also be used in an IPG, creating in this example a 17-electrode device (including the case).

Also shown in FIGS. 2A and 2B are DC-blocking capacitors 70 (Ci) placed in series between the electrodes nodes Ei′ present at the ASICs 60a and 60b and the electrodes 16 (Ei) appearing on the electrode array 12. The DC-blocking capacitors 70 act as a safety measure to prevent DC current injection into the patient, and are commonly used in IPGs. As shown in FIG. 2B, the DC-blocking capacitors 70 are ultimately coupled via the PCB 40 to electrode nodes Ei′ of the ASICs 60a or 60b, such as its bond pads 63 or package pins.

Given the amplitudes of the currents typically provided to the electrodes 16 for effective therapy (on the order of milliAmps), and given the desire to prevent large voltages from building up across the DC-blocking capacitors 70 when passing such currents, the DC-blocking capacitors 70 have relatively large capacitance values—typically on the order of 1-10 microFarads (μF), as explained further below. The DC-blocking capacitors 70 are thus relatively large in physical size, and can take up considerable space within the case 30 of the IPG 10, and in particular on the IPG's PCB 40.

This is shown in FIG. 3 for the 33-electrode IPG 10 described earlier, which shows the bottom surface of PCB 40 to which the charging coil 44 is mounted. Also mounted to the PCB 40 within the charging coil 44 are the ASICs 60a and 60b (each supporting 16 electrodes, and one supporting the case electrode 30), the microcontroller 62, and the DC-blocking capacitors 70. As one skilled in the art will appreciate, the PCB 40 would contain other components as well, but these are not shown for simplicity. The DC-blocking capacitors 70 typically comprise surface-mountable ceramic capacitors, each of which includes a component body 72 and solderable terminals 74 for coupling to the PCB 40. Such DC-blocking capacitors 70 may have areas (footprints on the PCB 40) of 120×60 mils (what is known in the art as a “1206” capacitor) (3.05×1.52 mm), 80×50 mils (an “0805” capacitor) (2.03×1.27 mm), or smaller.

Taken together, the DC-blocking capacitors 70 take up a relatively large area 80 on the bottom surface of the PCB 40, which may be as high as 30% of the total area of that surface (typically on the order of 10 cm²). This is unfortunate, because the DC-blocking capacitors 70 increase the size of the PCB 40, which increases the size of the case 30 and the IPG 10, which is preferably kept as small as possible to ease implantation surgery and promote patient comfort.

The problem of the size of the DC-blocking capacitors 70 is further exacerbated by the industry's desire to provide IPGs with greater number of electrodes to provide patients with finer resolution and more-complex stimulation therapies. While at least some of the DC-blocking capacitors 70 could theoretically be placed on the other (top) surface of the PCB 40, this is not always an option: for example, in the example of IPG 10, the top surface of the PCB 40 is already fully occupied by the communication coil 42, the battery 36, and other surface-mounted circuitry 50a, as shown in FIG. 1B.

The prior art has recognized the problem of relatively-large DC-blocking capacitors 70 in a multi-electrode IPG. However, known solutions to this problem seek to minimize the number of DC-blocking capacitors 70 used. For example, in U.S. Pat. No. 7,881,803, an IPG is provided having only a single DC-blocking capacitor, which saves room in the IPG and hence allows for IPG miniaturization. U.S. Patent Application Publication 2010/0268309 likewise proposes an IPG with a reduced number of DC-blocking capacitors, i.e., less than the number of electrodes the IPG supports. See also U.S. Patent Application Publication 2005/0245970.

However, “DC-blocking capacitor minimization” as taught in these prior art references is not viewed by the inventor as always providing a sufficient manner for addressing this problem. If too many DC-blocking capacitors 70 are removed compared to the number of electrodes the IPG supports, difficulties can arise. Therapy can be compromised, as the ability to freely choose electrodes operable as the anodes or cathodes may be restrained, because certain selectable current paths may not include a DC-blocking capacitor 70 as desired for safety. Additional design complexities of the current distribution circuitry must be undertaken to address this concern, such as by including switch matrices to allow the capacitors 70 to be shared. By contrast, if too few DC-blocking capacitors 70 are removed compared to the number of IPG electrodes 16, space savings in the IPG may be insignificant.

The problem of component size in an IPG is further exacerbated by newer advents that seek to add additional components to the IPG. For example, in U.S. Patent Application Publication 2014/0155970, it is taught to use additional Electromagnetic Interference (EMI) filtering capacitors 76 (C(f)) in the electrode current paths 64, as shown in FIGS. 4A and 4B. These filtering capacitors 76 are coupled to the electrode current paths 64 in parallel between each of the electrodes Ei and a reference voltage (e.g., ground, such as the battery 36's negative terminal). As taught in the 2014/0155970 Publication, filtering capacitors 76 are useful to shunt EMI coupled to the lead wires 20 to ground, such as the 64 MHz or 128 MHz frequencies typically present in a Magnetic Resonance Imaging (MRI) machine, and to hinder EMI from conducting to the conductive case 30. This reduces heating, and helps to protect the IPG circuitry from damage and from inadvertently stimulating the patient, thus making the IPG safer for use with patients requiring MRI procedures. Although not shown in FIGS. 4A and 4B, the 2014/0155970 Publication teaches that the filtering capacitors 76 could also be coupled on the other side of the DC-blocking capacitors 70, i.e., in parallel between the electrode nodes Ei′ and ground. Also disclosed in the 2014/0155970 Publication is the use of EMI filtering inductors 78 (Li), which like the DC-blocking capacitors 70 can placed in series in the electrode current paths 64, as shown in FIG. 4A, which filtering inductors Li can range from 0.5 to 3.0 μH. Alternatively, a single filtering inductor (Lcase) can be placed in the electrode current path of the case 30 having a value in the range of 50 to 200 nanoHenries (nH), as shown in FIG. 4B. Nonetheless, accompanying such additional components 76 and 78 is difficult, particularly when the space and area required for the DC-blocking capacitors 70 in the IPG 10 is considered.

A solution is therefore desired that reduces the size and area of the DC-blocking capacitors 70, preferably without the need to reduce their number relative to the number of electrodes the IPG supports. Such solutions are provided in this disclosure.

### BRIEF DESCRIPTION OF THE DRAWINGS

FIGS. 1A and 1B show different views of an implantable medical device, specifically an Implantable Pulse Generator (IPG) having a number of electrodes, in accordance with the prior art.

FIGS. 2A and 2B show current distribution circuitry for an IPG, including the use of DC-blocking capacitors in electrode current paths, in accordance with the prior art.

FIG. 3 shows a printed circuit board (PCB) for an IPG including the DC-blocking capacitors, in accordance with the prior art.

FIGS. 4A and 4B show the inclusion of filtering capacitors and filtering inductors in the electrode current paths, in accordance with the prior art.

FIG. 5 shows the need for a relatively-high DC-blocking capacitance in an IPG, in accordance with the prior art.

FIGS. 6A and 6B show improved circuitry for an IPG, including smaller-value DC-blocking capacitors, switching networks to alter the direction of current flow through the capacitors during a pulse, and electrode current path control circuitry for controlling the switching networks.

FIGS. 7A and 7B show further details of the improved circuitry, and illustrates operation of such circuitry when a biphasic pulse is passed through a smaller-value DC-blocking capacitor. FIG. 7C shows circuitry for measuring active charge recovery during the second phase of the biphasic pulse to render such charge recovery perfect. FIG. 7D shows use of the improved circuitry with biphasic pulses of random and varying current amplitudes and with different pulse widths during the first and second pulse phases.

FIGS. 8A and 8B show passive charge recovery circuitry useable in the prior art and with the improved circuitry respectively.

FIGS. 9A and 9B show use of the improved circuitry to measure the DC-blocking capacitances (FIG. 9A) and to use those measured capacitances to determine the current through the capacitances during normal IPG operation (FIG. 9B).

FIG. 10 shows an alternative architecture in which more of the improved circuitry is integrated within an ASIC chip with other current distribution circuitry.

FIGS. 11A-11C show the printed circuit board (PCB) in the IPG and resulting space savings when the smaller-value DC-blocking capacitors are used, and further show different means of incorporating the capacitors in the PCB.

### DETAILED DESCRIPTION

As discussed in the Background, DC-blocking capacitors are desirable to use in an IPG for safety reasons, and it is particularly desirable to use a DC-blocking capacitor with each electrode supported by the IPG 10 to allow any electrode to be freely chosen to provide a therapeutic stimulation current without unduly complicating the design of the device.

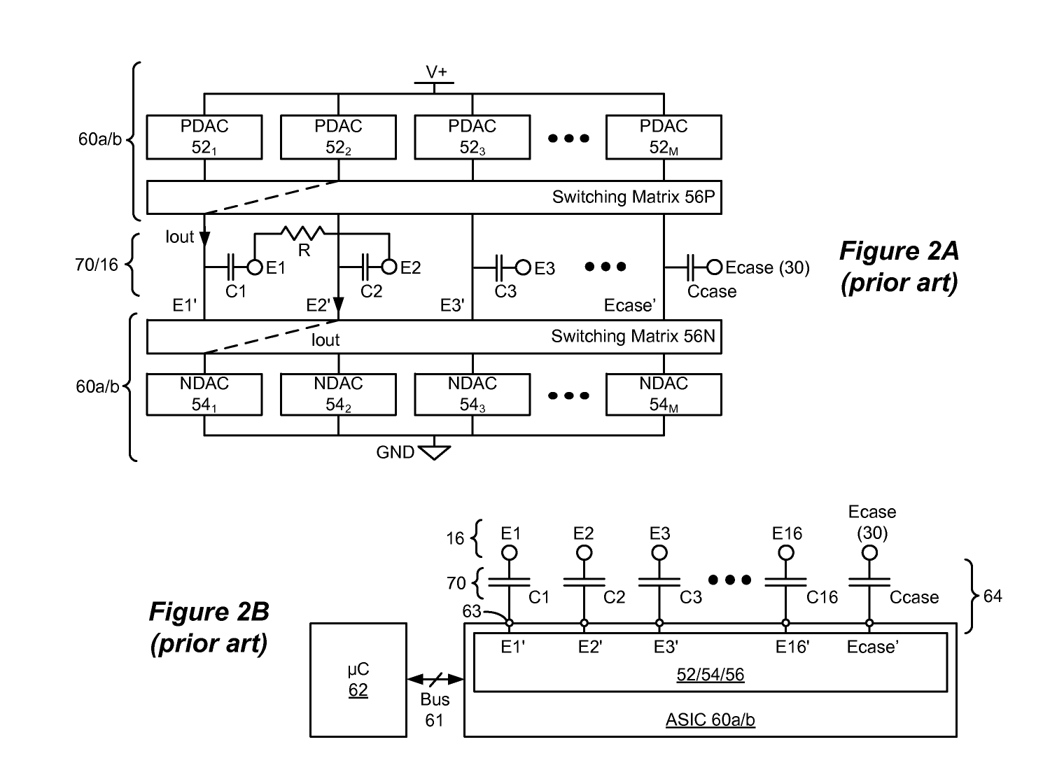

Unfortunately, these DC-blocking capacitors must have a relatively high capacitance, and thus a large size that takes up significant space on the IPG's PCB. The requirement of a relatively high capacitance for the DC blocking capacitors can be appreciated when considering a high-energy (“worst-case”) stimulation current pulse providable by an IPG 10. Consider for example a stimulation current pulse Iout with relatively long pulse width (PW) (e.g., 1 ms) of a relatively high constant-current amplitude (A) (e.g., 10 mA), as shown in FIG. 5. In the example shown, Iout is sourced to a patient's tissue R from anode electrode Ei using current source PDAC 52q, and sunk from the tissue at cathode electrode Ej using current sink NDAC 54r. (These DACs 52q and 54r may be coupled to electrodes Ei and Ej via the switch matrices 56P and 56N (FIG. 2A), although this detail isn't shown in FIG. 5).

When passing Iout through the DC blocking capacitor Ci associated with electrode Ei, charge, and hence voltage (Vi), will gradually build up across that capacitor during the pulse in accordance with the equation:

A=Ci*ΔVi/Δt (Eq. 1)

If electrode Ej also includes a DC-blocking capacitor 70, as is preferable for safety, but not strictly necessary, voltage (Vj) will also build up across that capacitor Cj.

While voltage build up Vi (and possibly Vj) across the DC-blocking capacitors Ci (and Cj if present) is inevitable, such voltage build up is preferably relatively small compared to the compliance power supply voltage V+ used to power the DACS 52/54, which voltage V+ is typically 5 to 15 Volts. Generally speaking, it is desirable that Vi (and Vj if capacitor Cj is present) not exceed a magnitude of 1V to allow sufficient headroom for V+ to power the DACs 52/54 so that they can reliably output the prescribed current amplitude A.

For the high-energy pulse Iout depicted in FIG. 5 (A=10 mA; Δt=1 ms), and assuming that ΔVi≦1V, the above equation informs that the capacitance Ci of the DC-blocking capacitor should be no less than 10 μF, which again is relatively large in size.

The issue of DC-blocking capacitor size cannot be easily addressed by merely lowering the value of its capacitance. For example, although not depicted, a 1 μF DC-blocking capacitor would in the above example yield a voltage across the capacitor Ci of Vi=10V at the end of the pulse (or a sum of 20V across both DC-blocking capacitors Ci and Cj if the latter is present). A voltage (sum) of this magnitude is on par with or exceeds the magnitude of the compliance power supply voltage V+. Thus, if DC-blocking capacitors of 1 μF are used in the IPG 10, its DACs 52/54 may be unable to provide the desired and programmed current amplitude (A) throughout the entirety of the pulse width, especially nearer to the end of the pulse, and especially for higher-energy pulses.

Despite these challenges, the inventor has devised improved circuitry 100 to allow much smaller-value DC-blocking capacitors to be used—with capacitance values orders of magnitude smaller than those used traditionally in an IPG 10. When such improved circuitry 100 is used, the DC-blocking capacitors can be 10 nF in one non-limiting example. As will be discussed subsequently, such smaller-value DC-blocking capacitors do not take up significant space in the IPG, or surface area on the IPG's PCB. Additionally, the improved circuitry includes the ability to measure the current amplitude provided to selected electrodes—for example, to ensure that the DACs are actually providing the prescribed current amplitude to the patient's tissue—and to provide for perfect active charge recovery.

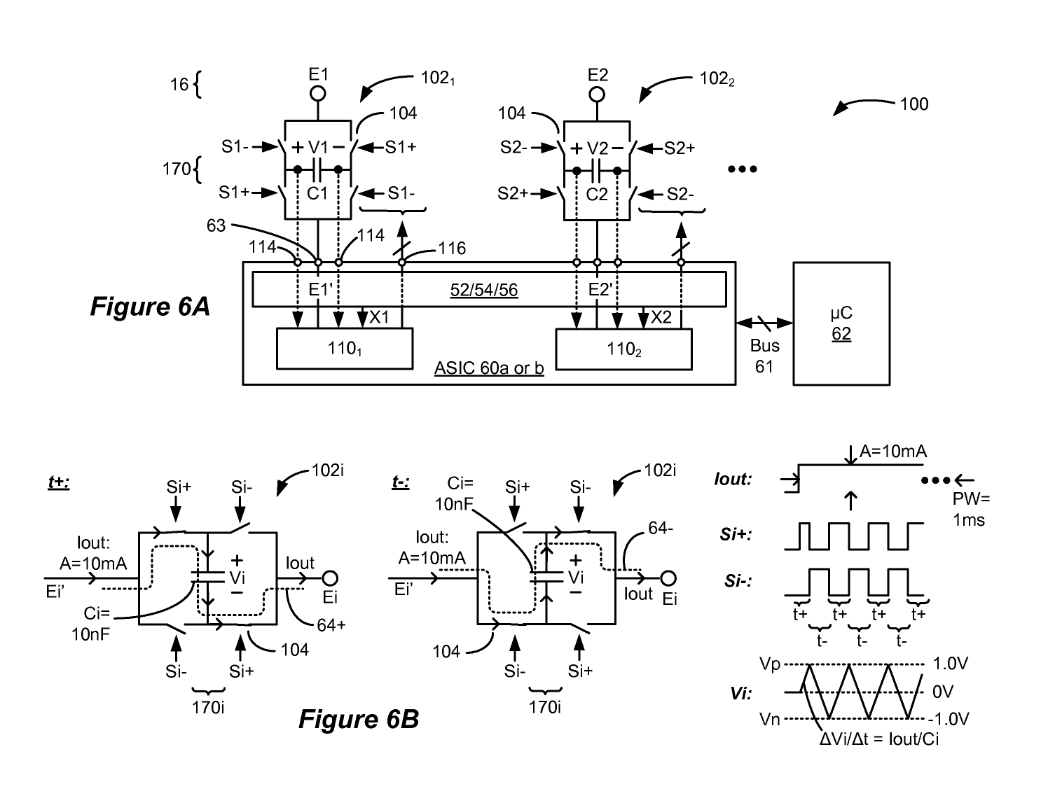

A first example of improved circuitry 100 is shown in one example in FIG. 6A. Much of the current distribution circuitry (PDACs 52, NDACs 54, switch matrices 56P and 56N) can be as described earlier, and depicted aspects unchanged from FIGS. 2A and 2B retain the same element numerals. New to the circuitry are the DC-blocking capacitors 170, which are smaller in value and size (e.g., 10 nF) compared to their counterparts 70 discussed earlier (e.g., 10 μF).

Each of the DC-blocking capacitors 170 is provided within a switching network 102 in an H-bridge configuration with four switches 104. The switches 104 in the switching networks 102 are controlled by electrode current path control circuitry 110, which as shown can be designed into and provided by the ASICs 60a and 60b. As shown, there are one switching network 102i and one electrode current path control circuit 110i associated with each DC-blocking capacitor Ci and electrode Ei. However, each electrode/capacitor need not necessarily have its own electrode current path control circuit 110, and a smaller number of such circuits 110 can be shared, which is sensible as only a few electrodes are typically active to provide a stimulation current pulse at any given time.

Prior to discussing the details of electrode current path control circuitry 110, basic operation of the switch network 102i is shown in FIG. 6B for an electrode Ei selected to source or sink a current pulse, Iout. (Operation and circuitry for electrode Ej is not shown, but would operate similarly if it includes capacitor Cj). The goal of the switch network 102i is to alternate the direction of current flow through the smaller-value DC-blocking capacitor Ci during the pulse, i.e., during its prescribed pulse width PW. In this regard, the electrode current path control circuit 110i issues two control signals Si+ and Si− to the switches 104 in the switch network 102i, which control signals are preferably complementary to close only two of the four switches 104 at a time.

When Si+ is asserted at a given times t+ during the pulse, two of the four switches 104 are closed, and the current Iout travels in a first direction through the DC-blocking capacitor Ci via electrode current path 64+. This causes a voltage Vi to build up across the capacitor Ci, as shown in the timing diagrams to the right in FIG. 6B. Because the capacitance Ci is relatively small, the voltage Vi will build at a relatively quick rate (i.e., with a high slope: ΔVi/Δt=A/Ci).

When this voltage Vi builds to an unacceptably-high upper threshold level (e.g., Vi=Vp=1.0V), Si− is asserted at times t− to close the other two of the four switches 104, thus changing the direction (second direction) of the current through the capacitor 170i via current path 64−. This will cause Vi to fall, again with a rate dictated by the capacitance Ci. Eventually all previous stored charge will be recovered (Vi=0V), and if Si− continues to be asserted, Vi will eventually be drawn to an unacceptably-low lower threshold level (Vi=Vn=−1.0V), at which point control signal Si+ is again asserted.

The result as shown is that the voltage Vi across the DC-blocking capacitor Ci will vary in a sawtooth fashion between the two thresholds Vp and Vn. Yet, the therapeutic current pulse Iout is still provided from the electrode node Ei′ in ASIC 60a or 60b to the selected electrode Ei in uninterrupted fashion during the pulse width PW and with the prescribed amplitude A.

Switches 104 may comprise single transistors, transfer gates, or more complicated switching circuits having negligible resistances and/or negligible voltage drops across them. Note that some switch 104 designs (e.g., transfer gates) require both a true (Si+) and complementary (Si−) control signal, and so all switches 104 may receive both signals, as one skilled in the art will understand, although this is not shown for simplicity.

In the example shown in FIG. 6A, the DC-blocking capacitors 170 and their associated switching networks 102 are “off chip,” i.e., are not implemented within the ASICs 60a/b. Accordingly, ASICs 60a/b include additional bond pads 114 that couple to both sides of the DC-blocking capacitors 170, and one more bond pads 116 for carrying the control signals Si+ and Si−. However, this architecture is not strictly necessary and the DC-blocking capacitors 170 and their associated switching networks 102 can involve different levels of integration with the ASICs 60a/b, as described later.

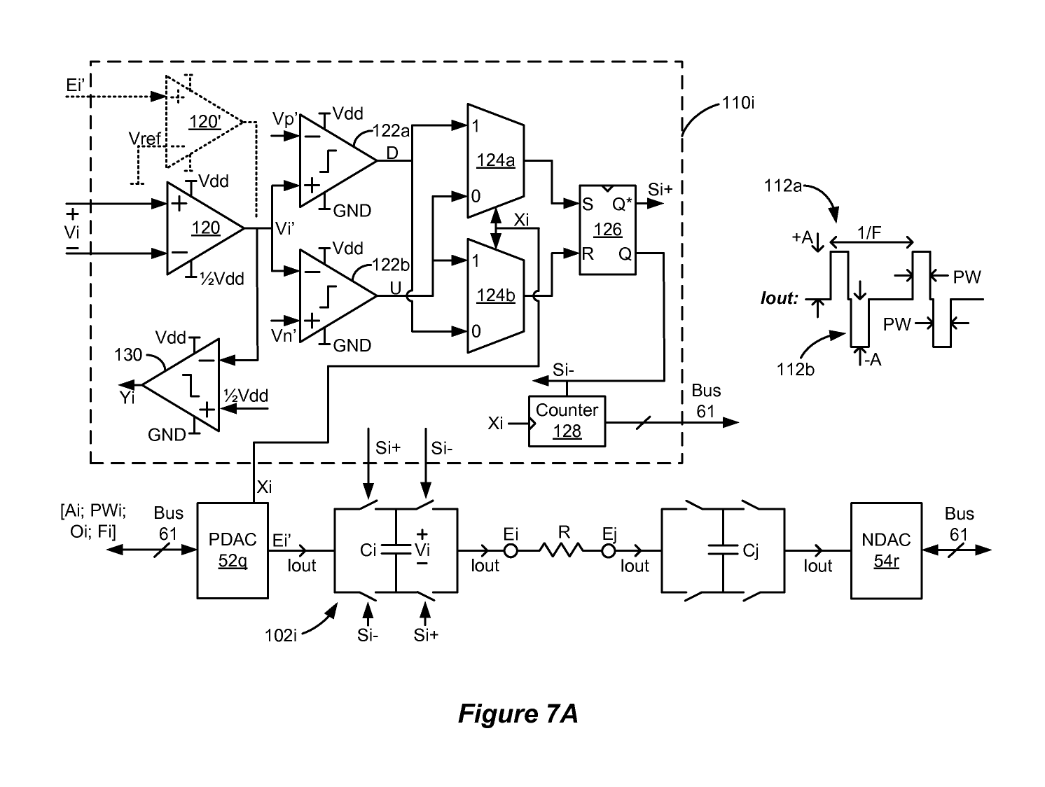

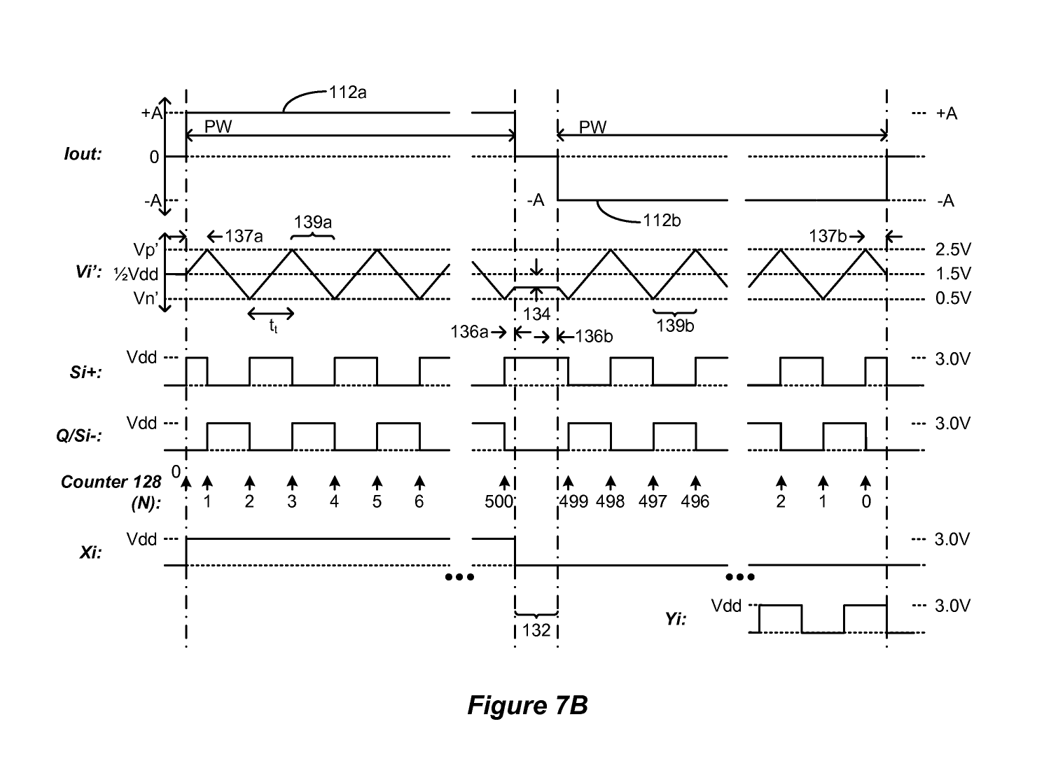

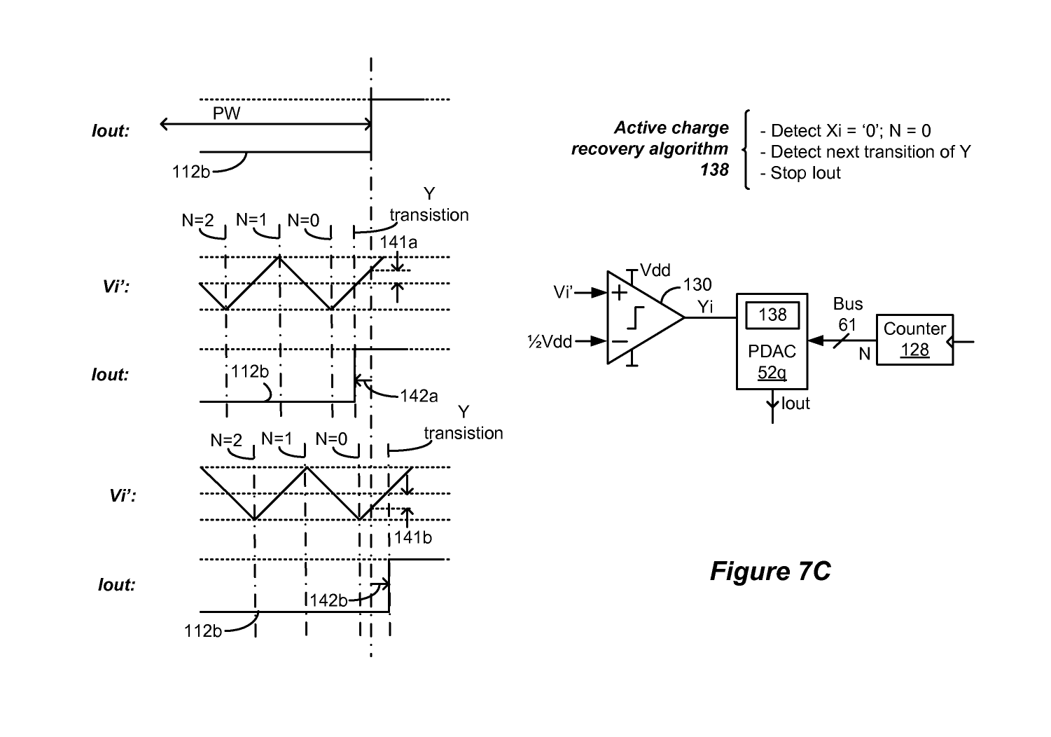

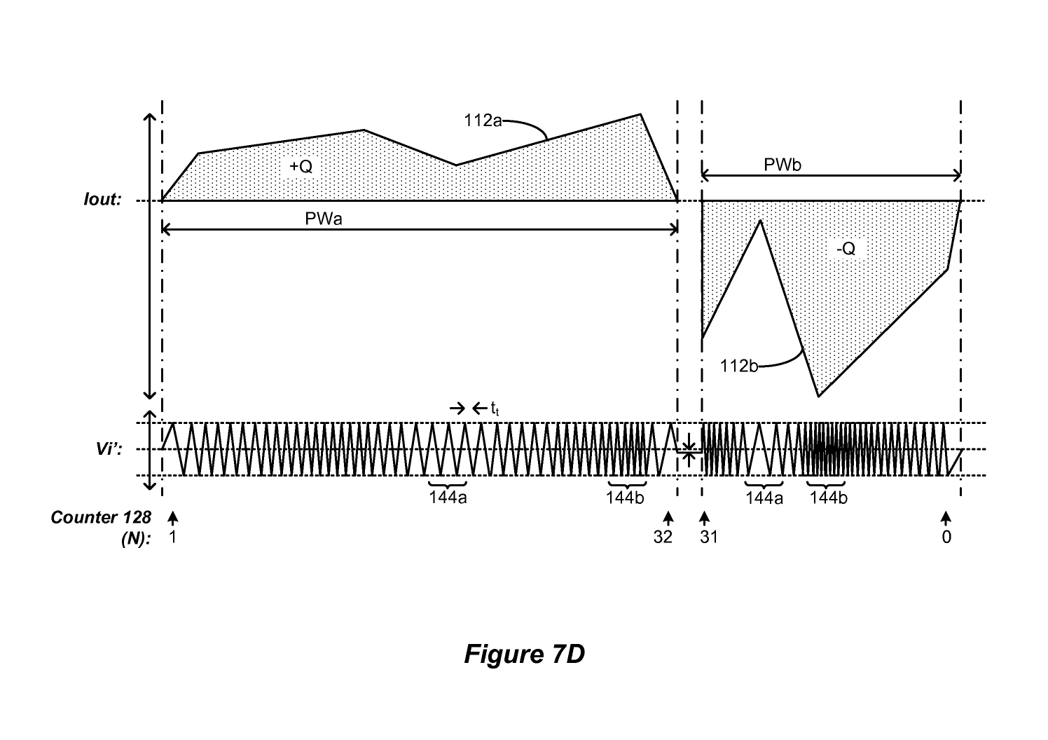

FIG. 7A shows further details of an example of the electrode current path control circuit 110i used to monitor the voltage Vi across a given DC-blocking capacitor Ci, and to issue the control signals Si+ and Si− to that capacitor's switch network 102i. Such details are explained in the context of issuing a biphasic current pulse Iout to the electrode Ei associated with capacitor Ci, which pulse is shown in the timing diagram to the right in FIG. 7A, and in greater detail in the timing diagram of FIG. 7B. The biphasic current pulse Iout comprises a pulse with two phases: a first phase 112a of a prescribed current amplitude (A), pulse width (PW), and polarity (O) indicating whether the first phase should be anodic (O=‘1’) or cathodic (O=‘0’). This is followed by a second phase 112b, which in the depicted example has nominally the same pulse width (although this is discussed in further detail below), but an amplitude of opposite polarity (−A).

As is well known in the IPG arts, the use of biphasic pulses is beneficial as a means of actively recovering any charge that might reside at the end of a pulse. That is, any charge that is stored on the DC-blocking capacitors 170 and other parasitic capacitances (e.g., at the electrode/tissue interface; not shown) during the first pulse phase 112a is actively recovered by driving a second pulse phase 112b having the same total charge (A*PW) as the first 112a, but of opposite polarity.

Electrode Ei is depicted as coupled (at least initially) to a particular PDAC 52q, and a corresponding sink electrode Ej is depicted as coupled to a particular NDAC 54r, perhaps via the switching matrices 56N and 56P (not shown) discussed previously (FIG. 2A). Description of the electrode current path control circuit 110 and switching network 102 is directed to electrode Ei and its DC-blocking capacitor Ci, but electrode Ej and capacitor Cj may also include and be controlled by such circuitry.

The pulse parameters for electrode Ei (Ai; PWi; Oi) may be provided by bus 61 from the microcontroller 62 to the PDAC 52q, along with the frequency that such pulses should issue (Fi). Notice that the PDAC 52q provides a pulse phase control signal Xi, shown in the timing diagram of FIG. 7B, indicating whether the circuitry is currently issuing the first phase 112a of the biphasic current pulse (Xi=‘1’) or the second phase 112b (Xi=‘0’). Pulse parameters may also be provided to NDAC 54r to allow electrode Ej to act as the corresponding sink or source for the current provided by electrode Ei.

Electrode current path control circuit 110i for electrode Ei includes a differential amplifier 120 for monitoring the voltage Vi across DC-blocking capacitor Ci during the pulse, which voltage may be fed back to the ASIC 60a or 60b via bond pads 114 discussed earlier (FIG. 6A). Because voltage Vi can be negative, the differential amplifier 120 shifts the DC level of Vi around a midpoint voltage to render an output Vi′ that is always positive and thus more easily handled by the circuitry in the electrode current path control circuit 110i. For example, assume the control circuitry 110i is powered by a power supply voltage Vdd=3.0 V in the ASIC 60a or 60b. Differential amplifier 120 may be provided a midpoint reference voltage of ½Vdd=1.5 V, thus producing a shifted capacitor voltage Vi′ varying around this midpoint, as shown in the timing diagram of FIG. 7B.

Alternatively, the voltage across the capacitor may be monitored by differential amplifier 120′ using electrode node Ei′ on the ASIC 60a or 60b as an input, as shown in dotted lines in FIG. 7A. This alternative requires providing a suitable reference voltage Vref to the differential amplifier 120′ (such as ground), and is further complicated by the fact that the voltage of Ei′ will vary depending on the current provided, the resistance R of the patient's tissue, etc. Still, the voltage of Ei′ may nonetheless be monitored and effectively constrained to a threshold change (such as +/−1V) by the circuit 110i. Even though this is more complex, this alternative provides the benefit of reducing the number of bond pads (such as 114) that need to be added to the ACISs 60a and 60b.

The shifted capacitor voltage Vi′ is provided to a window comparator comprising comparators 122a and 122b. Further provided to these comparators 122a and 122b are upper and lower threshold voltages Vp′ and Vn′, which may also comprise level-shifted versions of the Vp and Vn thresholds discussed with reference to FIG. 6B, and so may equal 2.5V and 0.5V respectively in the depicted example. Generation of these reference voltages Vp′ and Vn′ can be accomplished using well-known adjustable band gap voltage generator circuits for example.

Comparators 122a and 112b output digital signals D and U representing whether it is desired to lower (Down) or raise (Up) Vi′ via the control signals Si+ and Si−. These digital signals D and U are provided to multiplexers (muxes) 124a and 124b, whose control input is coupled to the pulse phase control signal Xi discussed earlier. The outputs of muxes 124a and 124b are provided to a latching SR flip flop 126, whose output Q comprises control signal Si−, and whose complementary output Q* comprises control signal Si+. Output Q (and/or Q*) is also provided to a counter 128, which counts the number of Q or Q* transitions, as explained further below. In a preferred example, the counter 128 can be controlled by the pulse phase control signal Xi to increment when Xi=‘1’ during the first pulse phase 112a, and to decrement when Xi=‘0’ during the second pulse phase 112b, for reasons explained further below. Comparator 130 is explained further below.

With the circuit elements of the electrode current path control circuit 110i so introduced, its operation is now explained, with continuing reference to the timing diagrams of FIG. 7B. Included in this explanation is a discussion of how the improved circuitry not only provides active charge recovery via use of the second pulse phase 112b, but additionally measures the total charge during both of pulse phases 112a and 112b to ensure that they are equal. Measuring charge is accomplished by counting the transitions (N) in the direction of the current through the DC-blocking capacitor during both of the pulse phases 112a and 112b. As will be explained further below, measuring the total charge during both phases 112a and 112b allows the pulse width of the second phase to be adjusted from its nominal value is necessary to ensure active charge recovery is perfect.

At the start of first pulse phase 112a, Iout issues with an amplitude of +A, and Xi=‘1’ indicating issuance of the first pulse phase 112a at electrode Ei. At this point, Vi=0V, and shifted capacitor voltage Vi′=1.5 V, which is between Vp′ and Vn′. Comparators 122a and 122b thus set signals U and D to ‘0’, which are passed by the top inputs of muxes 124a and 124b (Xi=‘1’) to the inputs of SR flip flop 126 (S=‘0’; R=‘0’). SR flip flop 126 thus outputs Q=Si−=‘0’, and Q*=Si+=‘1’. Thus, Iout is sent in the first direction through DC-blocking capacitor Ci via electrode current path 64+ as described earlier, which causes Vi and Vi′ to rise (137a).

Eventually, Vi′ will rise higher than Vp′=2.5V, and thus comparator 122a will assert signal D=‘1’. The top input of mux 124a provides this asserted signal to the flip flop 126, which is set (S=‘1’), setting output Q=Si−=‘1’, and Q*=Si+=‘0’, thus sending Iout in the second direction through DC-blocking capacitor Ci via electrode current path 64− as described earlier, which causes Vi and Vi′ to fall. Along with this change in the direction of the current, and because Xi=‘1’, the counter 128 increments (N=1).

Eventually, Vi′ will fall below Vn′=0.5V, thus comparator 122b will assert signal U=‘1’. The top input of mux 124b provides this asserted signal to the flip flop 126, which is reset (R=‘1’), setting output Q=Si−=‘0’ and Q*=Si+=‘1’. The direction of current flow through the capacitor Ci is thus reversed, causing Vi and Vi′ to rise, and again incrementing the counter 128 (N=2). This process continues until the end of the first pulse phase 112a.

The number of times the direction of the current transitions through DC-blocking capacitor Ci during the pulse width (PW) can be expressed as:

N=PW*A/[Ci*(Vp′−Vn′)] (Eq. 2)

and so N should equal 500 for the pulse Iout described earlier, where A=10 mA, PW=1 ms, and assuming Ci=10 nF and the difference between thresholds Vp′ and Vn′ equals 2V (2.5−0.5). Lower amplitude constant currents or shorter pulse widths would thus yield smaller number of transitions (N) within each pulse width. The timing between each transition, tₜ, equals

tₜ=Ci*(Vp′−Vn′)/A (Eq. 3)

and so tₜ would equal 2 μs for this same pulse, with lower amplitude currents yielding higher values for tₜ.

At this point, and prior to assertion of the second pulse phase 112b, the DACs 52/54 may institute an interphase period 132 (FIG. 7B) during which no stimulation is provided. Interphase period 132 is generally short and allows time for the current distribution circuitry to reverse the polarity of Iout during the second pulse phase 112b. This may involve for example coupling PDAC 52q to electrode Ej and NDAC 54r to electrode Ei via switch matrices 56P and 56N (FIG. 2A).

Note in FIG. 7B that the DC-blocking capacitor Ci may not be completely discharged at the end of the first pulse phase 112a, as indicated by the residual voltage 134 shown during the interphase period 132 between Vi′ and its midpoint (e.g., ½Vdd). To ensure perfect active charge recovery during the second pulse phase 112b, it is preferred to operate the electrode current path control circuitry 110 symmetrically but in reverse during the second pulse phase 112b. That is, if Vi′ had been rising (136a) at the end of the first pulse phase 112a (Q=Si−=‘0’), it will begin falling (136b) at the beginning of the second pulse phase 112b, and vice versa. In this manner, the charge comprising the residual voltage 134 resulting from the fraction of a transition 136a at the end of the first pulse phase 112a is recovered at a corresponding fractional transition 136b occurring at the beginning of the second pulse phase 112b.

Such reverse operation of the circuitry is controlled using pulse phase control signal Xi, which will now equal ‘0’ during second pulse phase 112b. Because Xi controls the muxes 124a and 124b, Xi during the second pulse phase 112b reverses which of signals D and U are used to set and reset the flip flop 126. Additionally, output Q at the end of the first pulse phase 112a remains latched during the interphase period 132, and will affect the direction of the current through the capacitor Ci at the beginning of the second pulse phase 112b.

At the start of second pulse phase 112b, Iout issues with an amplitude of −A, and Xi=‘0’. At this point, Vi′ is between Vp′ and Vn′, although perhaps offset by the residual voltage 134, but nonetheless comparators 122a and 122b set signals U and D to ‘0’. Muxes 124a and 124b pass these values per their bottom inputs (Xi=‘0’) to the SR flip flop 126, which does not affect the previously-latched output Q of the flip flop 126. Thus, if voltage Vi′ had been rising at the end of the first pulse phase 112a as shown in FIG. 7B, output Q=Si−=‘0’ had been latched. Q*=Si+=‘1’, and thus Iout is sent in the first direction through DC-blocking capacitor Ci via electrode current path 64+ at the beginning of the second pulse phase 112b. However, because the magnitude of the current is of opposite polarity (−A) in the second pulse phase 112b, Vi and Vi′ fall, unlike operation during the first pulse phase 112a. By contrast, if voltage Vi′ had been falling at the end of the first pulse phase 112a (not shown), output Q=Si−=‘1’ had been latched, and Q*=Si+=‘0’, and thus current is sent in the second direction through DC-blocking capacitor Ci via electrode current path 64− at the beginning of the second pulse phase 112b. Again though, because the magnitude of the current Iout is of opposite polarity (−A) in the second pulse phase 112b, Vi and Vi′ would rise in this circumstance.

Regardless whether Vi′ initially rises or falls at the start of the second pulse phase 112b, it will eventually reach either upper threshold Vp′ or lower threshold Vn′. If Vi′ falls below Vn′=0.5V, signal U=‘1’ is asserted from comparator 122b. The bottom input of mux 124a provides this asserted signal to the flip flop 126, which is set (S=‘1’), setting output Q=Si−=‘1’, and Q*=Si+=‘0’. This sends Iout in the second direction, which causes Vi and Vi′ to rise (given Iout's opposite polarity). If Vi′ rises higher than Vp′=2.5V, signal D=‘1’ is asserted from comparator 122a. The bottom input of mux 124b provides this asserted signal to the flip flop 126, which is reset (R=‘1’), setting output Q=Si−=‘0’, and Q*=Si+=‘1’. This sends Iout in the first direction, which causes Vi and Vi′ to fall. In either case, the counter 128 detects the transition in current direction by virtue of the change in output Q. However, the counter 128 will now decrement during the second pulse phase 112b because Xi=‘0’. Thus as shown, if N=500 at the end of the first pulse phase 112a, it will be N=499 when the current first transitions during the second pulse phase 112b. Thereafter, and as before, Vi′ will again sawtooth between the thresholds Vp′ and Vn′ during second pulse phase 112b, with the counter 128 continuing to decrement.

Reverse symmetry in operation of the electrode current path control circuitry 110 and switching network 102 during the first and second pulse phases 112a and 112b suggests that residual charge (voltage) on the DC-blocking capacitor Ci (and by extension any parasitic capacitances) should be at zero at the end of the second pulse phase 112b, assuming ideal circuit operation, and even if a residual voltage 134 was present during the interphase period 132. That is, Vi should equal 0 Volts (or Vi′=½Vdd) at the end of the second pulse phase 112b as depicted in FIG. 7B.

However, this may not be true, and instead Vi may not be zero (Vi′ may not be ½Vdd) when the second pulse phase 112b is scheduled to end. For example, FIG. 7C shows two scenarios 141a and 141b in which Vi/Vi′ is too high 141a and too low 141b at the scheduled end of second pulse phase 112b. In other words, residual voltage 141a or 141b is present, and thus charge recovery is not perfect.

The improved circuitry addresses this issue by modifying the otherwise-scheduled end of the second pulse phase 112b via active charge recovery algorithm 138. As shown, the active charge recovery algorithm 138 is shown as circuitry and/or instructions inside of the PDAC 52q. This is not strictly necessary however. In the example shown, the active charge recovery algorithm 138 receives signal Yi from comparator 130 and the count N from the counter 128, which can be provided via bus 61 or otherwise. Pulse phase control signal Xi would already be known to the PDAC 52q.

Active charge recovery algorithm 138 begins by detecting when the count of the counter has decremented to N=0 during the second pulse phase 112b (Xi=0). This informs the PDAC 52q during the second pulse phase 112b that the current has now changed directions through the DC-blocking capacitor Ci the same number of times as occurred during the first pulse phase 112a. From a charge recovery standpoint, at this point, all complete transitions (139a, between Vn′ and Vp′) in the first pulse phase 112a have been recovered by the same number of complete transitions (139b, but of opposite polarity) during the second pulse phase 112b. And the fractional transition 136a at the end of the first pulse phase 112a has been recovered by the symmetrical fractional transition 136b at the beginning of the second pulse phase 112b, as described earlier.

All that remains is to recover the fractional transition 137a (FIG. 7B) at the beginning of the first pulse phase 112a via the fractional transition 137b at the end of the second pulse phase 112b. At this point then, the active charge recovery algorithm 138 merely needs to determine when there is no longer any residual voltage present on the capacitor Ci (Vi=0), and to stop issuance of the Iout pulse at that time. To assist in this determination, shifted capacitor voltage Vi′ is provided to comparator 130 which compares it to the midpoint reference voltage ½Vdd. Vi′ may be increasing or decreasing at this point, and so the active charge recovery algorithm 138 needs merely to monitor the point in time at which output Yi from the comparator 130 transitions state (from ‘0’ to ‘1’ or vice versa), and to stop Iout at that time. The result for scenario 141a is that the second pulse phase 112b will end earlier (142a) than nominally prescribed, with the effect that the pulse width 112b is smaller than the pulse width PW. The result for scenario 141b is that the second pulse phase 112b will end later (142b) than nominally prescribed, with the effect that the pulse width 112b is larger than pulse width PW.

Although illustrated as only stopping stimulation at PDAC 52q, it should be understood that asserting of signal Yi may be communicated to other DACs (e.g., using bus 61) involved in providing the current, such as NDAC 54r, so that all active DACs cease issuance of the current at the same time. Alternatively, active DACs, such as NDAC 54r, could stop their currents by independently running their own active charge recovery algorithms 138.

To summarize, the improved circuitry is able to effectively measure and guarantee that the total positive charge provided during the first pulse phase 112a of the biphasic pulse Iout equals the total negative charge provided during the second pulse phase 112b of the biphasic pulse, thus achieving perfect active charge recovery.

Integrating Equation 1 above informs that the charge (q) provided during a complete transition (139a or b; FIG. 7B) is

q=Ci*ΔVi′ (Eq. 4)

where ΔVi′=Vp′−Vn′=2V, and so q=10 nF*2V=20 nC. Notice that this amount of charge is independent of the amplitude (A) of the current.

Accordingly, and significantly, perfect active charge recovery can be measured and guaranteed for any biphasic pulse, even if the pulse phases 112a and 112b are not symmetric. This is illustrated in FIG. 7D, which shows a first pulse phase 112a and a second pulse phase 112b having: currents not of the same amplitude, and which may even vary; and pulse widths PWa and PWb of different durations. The total charge of the first pulse width 112a is +Q, which (ignoring the fractional transitions 136a/b and 137a/b for simplicity) equals N*q, or in the depicted example 32*20 nC or 64 nC. The total charge of the second pulse width 112b is the same, but of opposite polarity: −Q=−64 nC. The amplitude of the current Iout at any point during either pulse phase 112a or b only changes the frequency WO of the transitions of the direction of the capacitor current, with lower amplitude periods 144a resulting in lower frequency transitions, and higher amplitude periods 144b resulting in higher frequency transitions. Nonetheless, perfect active charge recovery is achieved, and Vi across the DC-blocking capacitor C1 equals 0 (Vi′=½Vdd) at the end of the second pulse phase 112b.

The ability of the improved circuitry to support the use of essentially random amplitudes and durations is beneficial. For example, while the DACs 52/54 have to this point been described as sources or sinks of constant current, they may now comprise variable current sources. Or, DACs 52/54 may comprise constant or variable voltage sources. The ability to use a voltage source for the DACs 52/54 is especially desirable. The exact current output by such a voltage source, Iout, may not be known, particularly if the resistance of the patient's tissue R is unknown or varies. Nonetheless, perfect active charge recovery can still be had by counting the number of transitions (N) of the direction of the capacitor current during both pulse phases 112a and 112b, even if the actual current is unknown or varies.

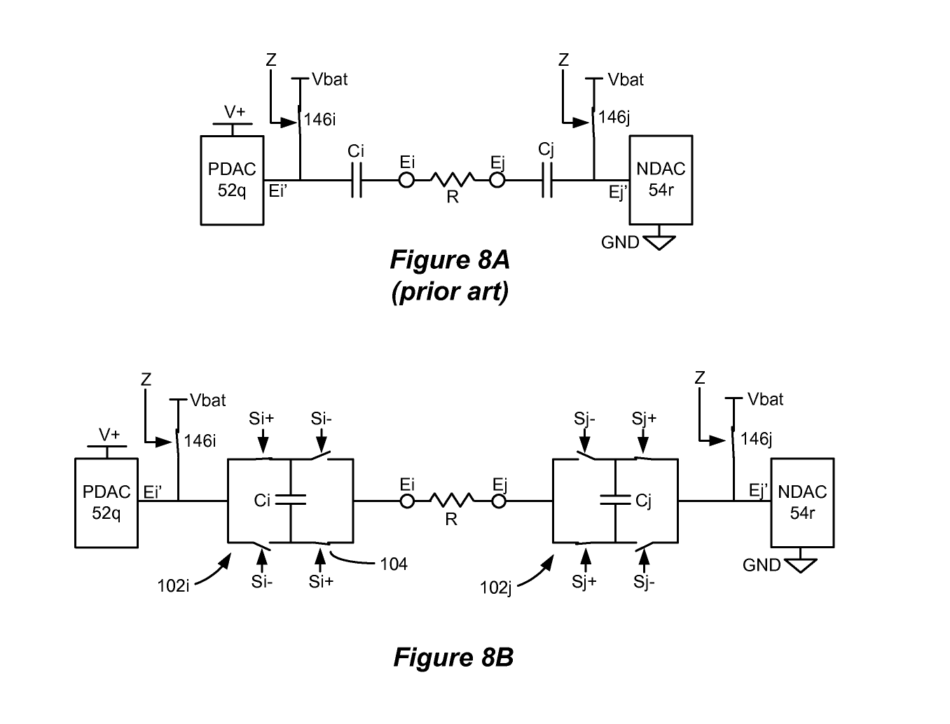

It is worth mentioning here that the art implicitly acknowledges that active means of charge recovery using a biphasic pulse may not be sufficient, and therefore that active charge recovery (i.e., use of an opposite-polarity second pulse phase 112b) can be followed by a passive charge recovery to equalize any remaining charge on capacitive structures. For example, as taught in U.S. Patent Application Publication 2013/0289661, and as shown in FIG. 8A, recovery switches 146 can be activated by a control signal Z after a biphasic pulse to couple previously-activated electrode nodes Ei′ and Ej′ (and alternatively, all electrode nodes in the IPG) to a common reference voltage. As shown, this reference voltage comprises Vbat, the voltage of the IPG battery 36, but could comprise other reference voltages as well, such as Vdd or ground for example.

Passive charge recovery can also occur after provision of a biphasic pulse using the improved circuitry, as shown in FIG. 8B, and again recovery switches 146 controllable by Z can couple between the electrode nodes Ei′ and Ej′ and the reference voltage. Additionally, preferably only one of the sets of switches 104 is closed in previously-activated or all switching networks 102, such as those controlled by Si+ and Sj+ for example, while the other set, Si− and Sj− remain open. This allows for passive charge recovery without directly communicating the reference voltage to the patient's tissue R. Passive charge recovery can also occur after a monophasic pulse as well. However, passive charger recovery should not be necessary with the improved circuitry and active charge recovery techniques discussed previously.

The improved circuitry is also useful in its ability to measure the amplitude of the current of a constant current pulse Iout, and to verify that constant-current DACs are actually outputting the correct current amplitude during a pulse. In effect, use of the counter 128 in conjunction with the electrode current path control circuitry 110 and switch networks 102 acts as a current meter. This is a beneficial addition to the IPG 10, because it cannot always be assumed that the DACs 52/54 are outputting programmed current amplitudes: for example, the compliance power supply value V+ may be too low, or the DACS 52/54 or other circuitry in the IPG 10 may be defective in some regard.

If the electrode current path control circuitry 110 and switch networks 102 are to act as a current meter, it can be useful to first determine precise values of the various DC-blocking capacitors 170. Note in this regard that the DC-blocking capacitors 170 may have a tolerance of +/−5% for example, or may range between 9.5 and 10.5 nF if nominally specified as 10 nF capacitors. Generally speaking, such variance is not problematic if the improved circuitry is merely used to generally reduce the capacitance values of the DC blocking capacitors 170. Nor is such variance problematic to practice of the active charge recovery algorithm 138 described earlier, because variation in the capacitance Ci will merely scale the number of transition N equally in both the first and second pulse phases 112a and 112b.

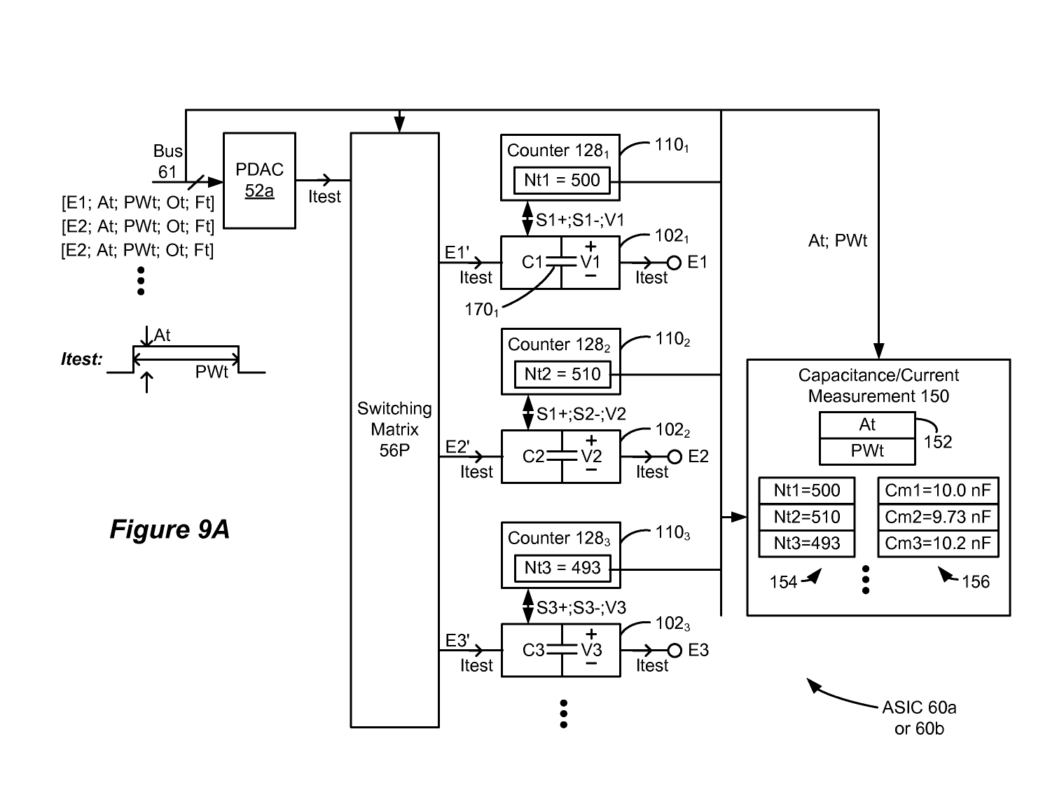

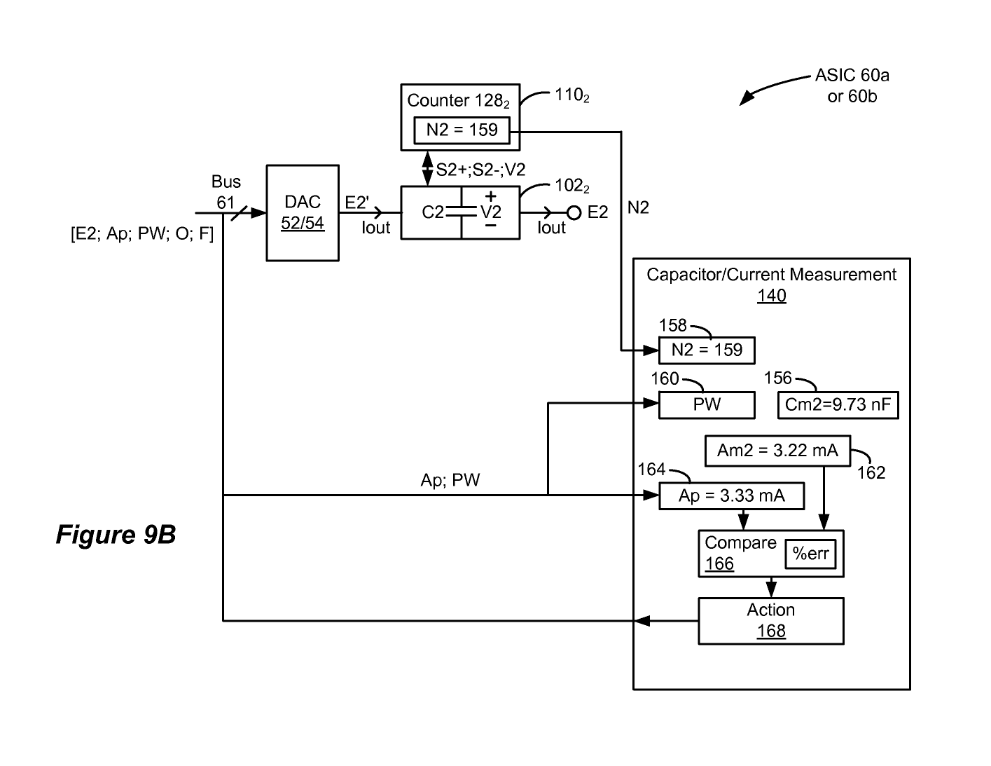

But if the improved circuitry is to be used to additionally measure a constant current output by the DACs 52/54, additional knowledge regarding the actual capacitance of each DC-blocking capacitor provides precision to the current measurement. In this regard, it is useful, although not strictly necessary, to determine the values for each of the DC-blocking capacitors 170, as shown in FIG. 9A. This can be done by providing a test current pulse, Itest, to each of the electrodes and by measuring the number of transitions (Nt) that occur during that pulse. From Nt, the capacitance of each DC-blocking capacitor 170 can be effectively measured (Cmi) and stored for subsequent use to measure therapeutic currents, as discussed in FIG. 9B.

The test current pulse Itest can comprise any current of constant amplitude (At) and pulse width (PWt) that will provide a number of transitions (Nt) commensurate with the desired accuracy of the measurement. Itest may comprise only a single monophasic pulse at each electrode Ei, although a train of monophasic pulses (allowing for multiple capacitance measurements at each electrode which could be averaged), a single biphasic pulse (with the capacitance measured during both of pulse phases 112a and 112b), or a train of biphasic pulses could be used as well.

The test current pulse Itest used to measure each DC-blocking capacitor 170 is preferably not uncomfortable for the patient, and may be sub-threshold relative to what the patient can sense. Alternatively, the capacitance of each DC-blocking capacitor 170 may be measured and stored in the IPG (e.g., in capacitance/current measurement module 150, discussed below) by the IPG manufacturer and prior to implantation in a patient. The manufacture can alternatively measure each of the DC-blocking capacitors Ci discretely during manufacturing using a tester, and store the measured values in the IPG (156) for use in measuring therapeutic currents later (FIG. 9B).

Provision of Itest can occur by providing test pulse parameters (specifying a particular electrode (Ei); a test amplitude (At); and a test pulse width (PWt)) to a given DAC via bus 61 as shown. (Polarity and frequency test pulse parameters Ot and Ft may not be needed if a single monophasic test current pulse is used). In the example shown, a single PDAC 52a is used to sequentially source the test current pulse Itest to each of the electrodes, with routing occurring via the switching matrix 56P described earlier. Although not shown, the test current pulse may also be sunk to the case electrode 30 (Ecase) acting as a ground. Using the same PDAC 52a to provide the test current pulse to each electrode is preferred to ensure the same magnitude At of current, because currents provided by different sources can vary. However, this is not strictly necessary if the PDACs 52 are well calibrated. Different PDACs may also be used, for example, in architectures having dedicated PDACs (and NDACs) at each electrode, as discussed earlier. While measuring the capacitances is illustrated using a source current, a sink current could be used as well—for example, by coupling each electrode to a given NDAC 54.

As each electrode Ei is provided the test current pulse, its electrode current path control circuitry 110i and its switch network 102i operate as described earlier, with its counter 128i determining the number of transitions (Nti) experienced during the pulse. These test numbers Nti for each electrode (Nt1, Nt2, etc.) are stored in file 154 in a capacitance/current measurement module 150. The capacitance/current measurement module 150 is preferably within the ASIC 60a/b, but alternatively may reside at the microcontroller 62. The capacitance/current measurement module 150 can know when to populate its count file 154 by communication with the bus 61, which bus may also inform it of the test amplitude (At) and pulse width (PWt) if such values are not already pre-populated in registers 152. Once the module 150 has received the test numbers (Nti) at each electrode, and knows the test amplitude and pulse width (At; PWt), it can determine (measure) the capacitance Cmi for the DC-blocking capacitor at each electrode by applying Equation 2 as rearranged:

Cmi=PWt*At/[Nti*(Vp′−Vn′)] (Eq. 5)

The resulting capacitance values are stored in file 156 for use in measuring later-provided therapeutic currents, as described next.

Once measured capacitance file 156 is populated, the capacitance/current measurement module 150 can use this file to measure the current used during the provision of an actual constant stimulation current Iout (during either of pulse phases 112a or 112b) to a patient, as shown in FIG. 9B. In this example, Iout is being provided to electrode E2. Module 150 can receive the programmed pulse amplitude (Ap) and pulse width (PW) that DAC 52/54 is expected to provide from bus 61, which parameters may vary depending on patient preferences. The programmed pulse amplitude and pulse width are stored in registers 164 and 160 respectively.

During provision of the constant-current pulse at electrode E2, the number of transition N2 is determined, and provided to a register 158 in the module 150. The module 150, knowing that the pulse was provided to electrode E2, can retrieve the previously-measured capacitance Cm2 of DC-blocking capacitor C2 from file 156. Once again rearranging Equation 2, a measured current amplitude Am2 at electrode E2 can be determined:

Am2=N2*Cm2*(Vp′−Vn′)/PW (Eq. 6)

and stored in register 162.

Note that the current measurement illustrated in FIG. 9B focuses on only one electrode E2 implicated in providing the therapeutic current, Iout. As one skilled in the art understands and as explained above, another electrode would be implicated in providing this current as well. Thus, this same measurement could be made at such other electrode(s). This is however not described for simplicity, and may be redundant, as the measured current should be the same, especially if only two electrodes are implicated to provide the therapeutic current.

Thereafter, module 150 can compare the measured current amplitude Am2 to the value the DAC 52/54 was expected to provide, i.e., the programmed amplitude Ap, which as noted earlier is stored in register 164. A comparison module 166 may compare Am2 and Ap to determine their difference, for example, expressed as an error percentage. Depending on this difference, an action module 168 may issue an appropriate response. For example, if the programmed Ap and measured Am2 constant current amplitudes differ by more than a percentage threshold (e.g., 3%), action module 168 may issue an appropriate action instruction.

Such an action instruction from the action module 168 is preferably issued to the master microcontroller 62 via bus 61, which may in turn take one or more of the following actions: (1) log the discrepancy for later interpretation; (2) instruct the IPG's telemetry circuitry to wirelessly transmit an appropriate indication of the discrepancy to an external controller device via communication coil 42; (3) issue an alarm; (4) instruct the ASIC 60a or 60b to use another DAC 52/54; etc. The microcontroller 62 may take additional actions as well. For example, if the measured current Am2 is lower than expected (per Ap), the microcontroller 62 may inform the ASICs 60a or 60b to increase the magnitude of the compliance power supply voltage V+ to provide additional power to the DAC 52/54 to rectify the shortfall.

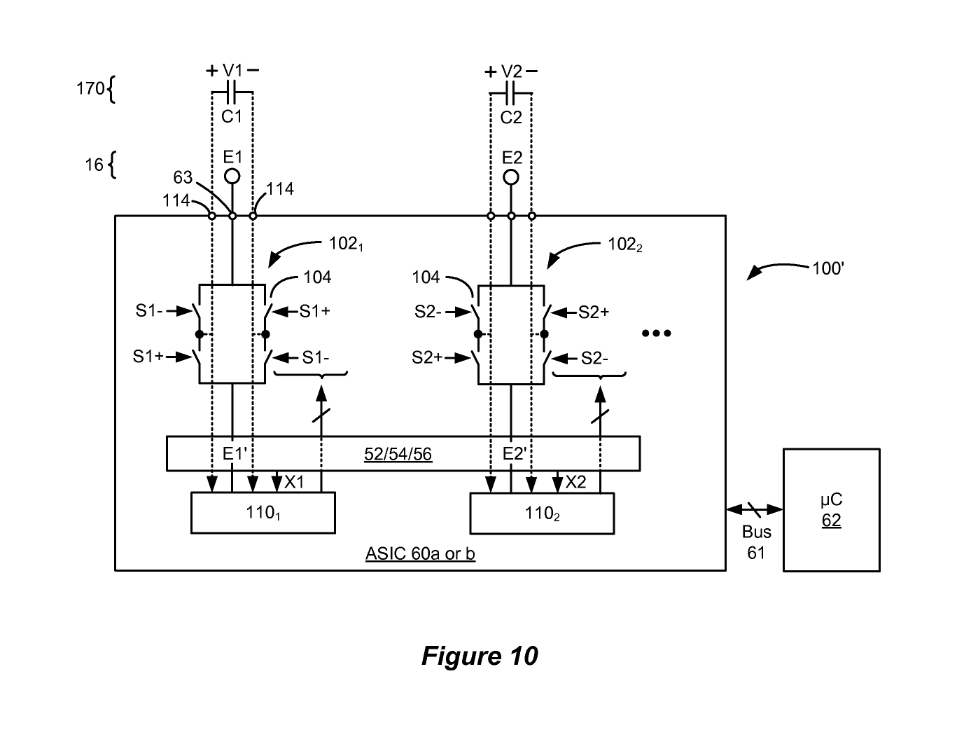

FIG. 10 shows a different implementation of the improved circuitry with further aspects integrated within the ASICs 60a/b. In particular, the switches 104 in the switches networks 102 are formed within the ASICs 60a/b, with the benefit that bond pad(s) 116 (FIG. 6A) are not required for routing of the control signals Si+ and Si−. In another example, and in light of the fact that the disclosed technique allows for the use of smaller capacitance values, the DC-blocking capacitors 170 may also be integrated within the ASICs 60a/b, thus providing a completely integrated solution, thus eliminating the need for bond pads 114 as well. However, it may be necessary in such an implementation to even further lower the capacitance of the DC-blocking capacitors (e.g., <10 nF), or to use fewer of them (using the “DC-blocking capacitor minimization” prior art techniques disclosed above), or to support fewer electrodes (<33), because it may be difficult to realize a suitable capacitance using the technology and area available in the ASICs 60a/b.

Notwithstanding the reduction in capacitance value, DC-blocking capacitors 170 can still be provided in series for each electrode 16 supported by the IPG 10, which as noted earlier prevents DC current injection into the patient and promotes patient safety. Because a DC-blocking capacitor 170 is preferably provided with each selectable electrode 16 (and case 30 electrode Ecase), the electrodes can be freely selected as anodes or cathodes without concern of DC current injection, and without complicating the design of the current distribution circuitry in the IPG 10, which can otherwise remain as illustrated in FIGS. 2A and 2B.

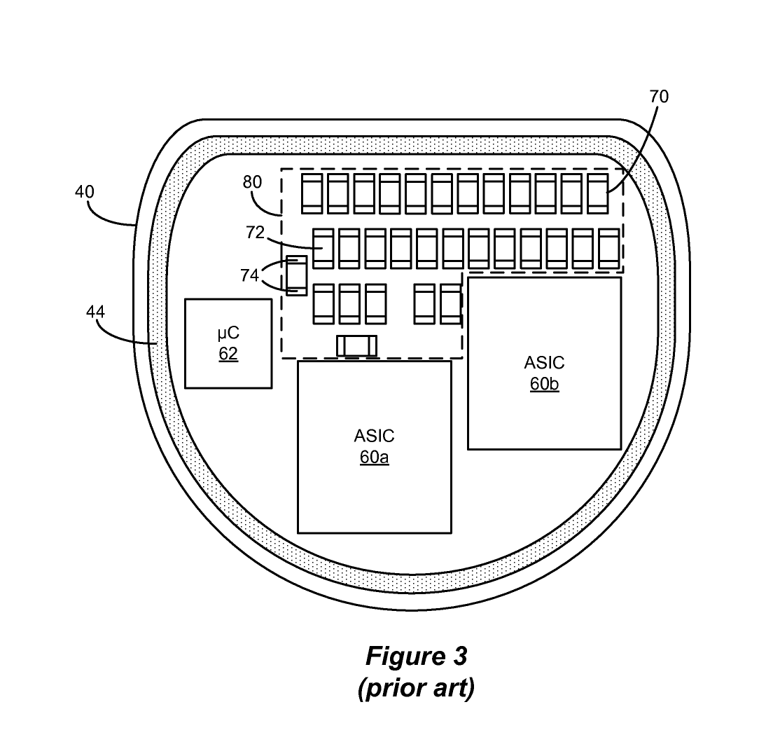

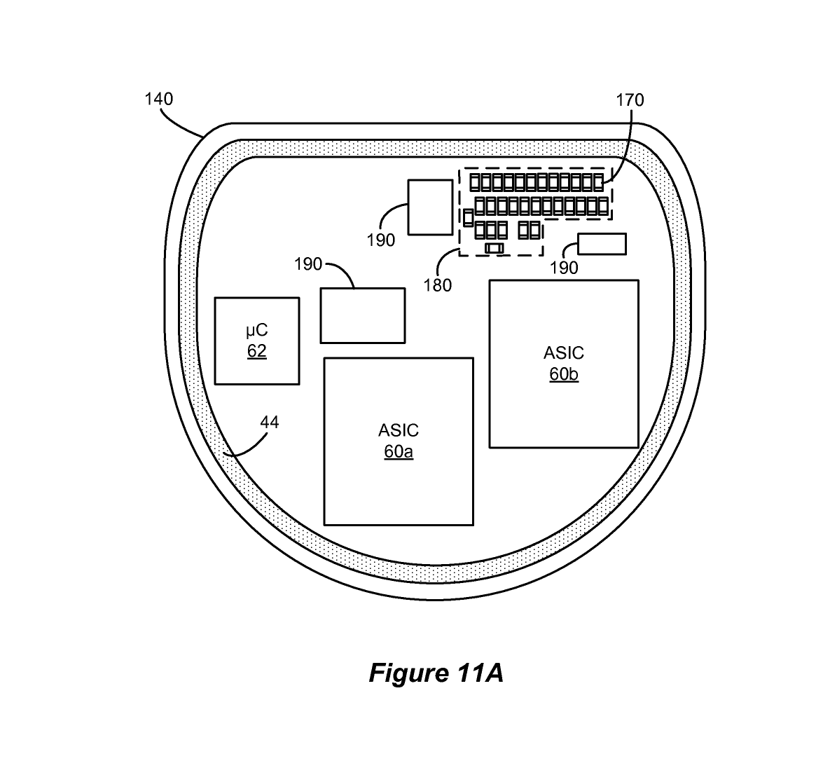

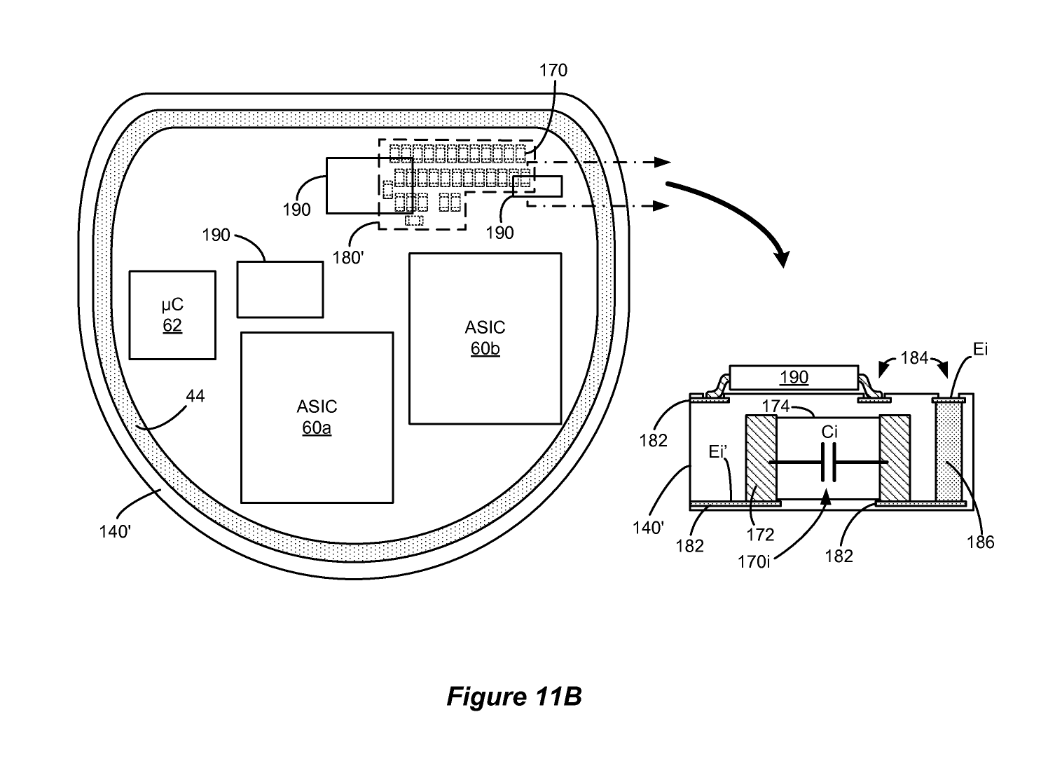

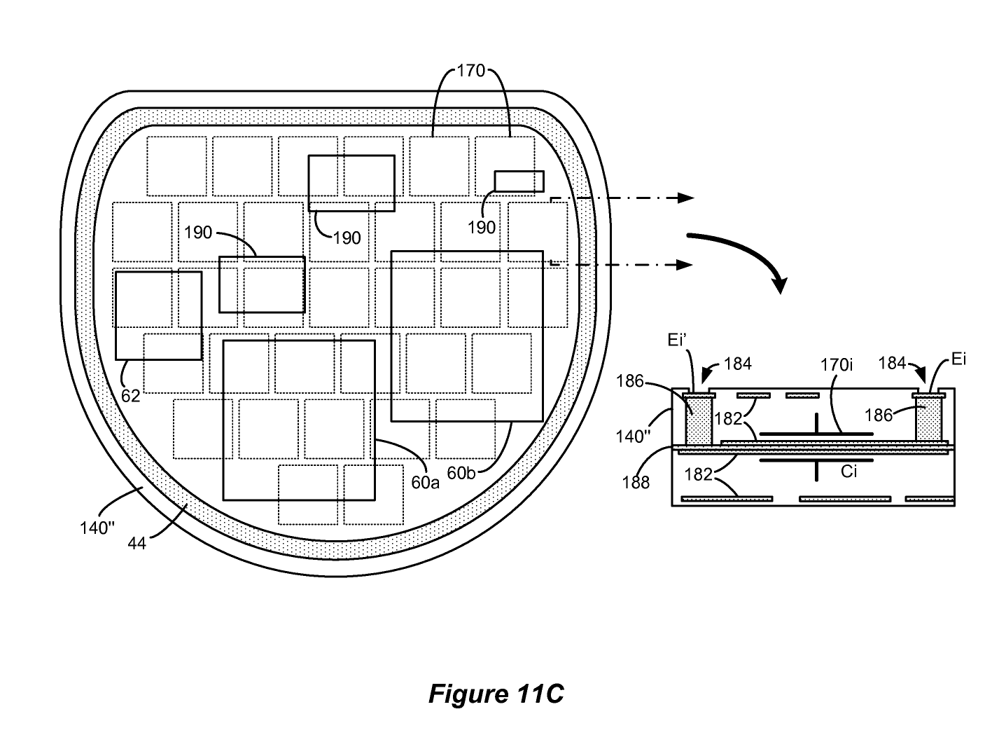

Because the DC-blocking capacitors 170 have smaller capacitance values, they can be of much smaller sizes than the DC-blocking capacitors 70 illustrated earlier. FIG. 11A shows a printed circuit board (PCB) 140 for use with IPG 10 having such smaller DC-blocking capacitors 170, which may comprise 0402 capacitors, and still-yet smaller 0201 capacitors, which are available at 10 nF for example. See, e.g., Part No. 0201ZC103JAT2A, supplied by AVX Corporation. The DC-blocking capacitors 170 take up a much smaller area 180 on the PCB 140 than do the DC-blocking capacitors 70 in area 80 on the PCB 40 of FIG. 3. As such, the PCB 140 can be made smaller than PCB 40, which allows the IPG 10 to be made smaller, promoting patient comfort and easing implantation concerns.

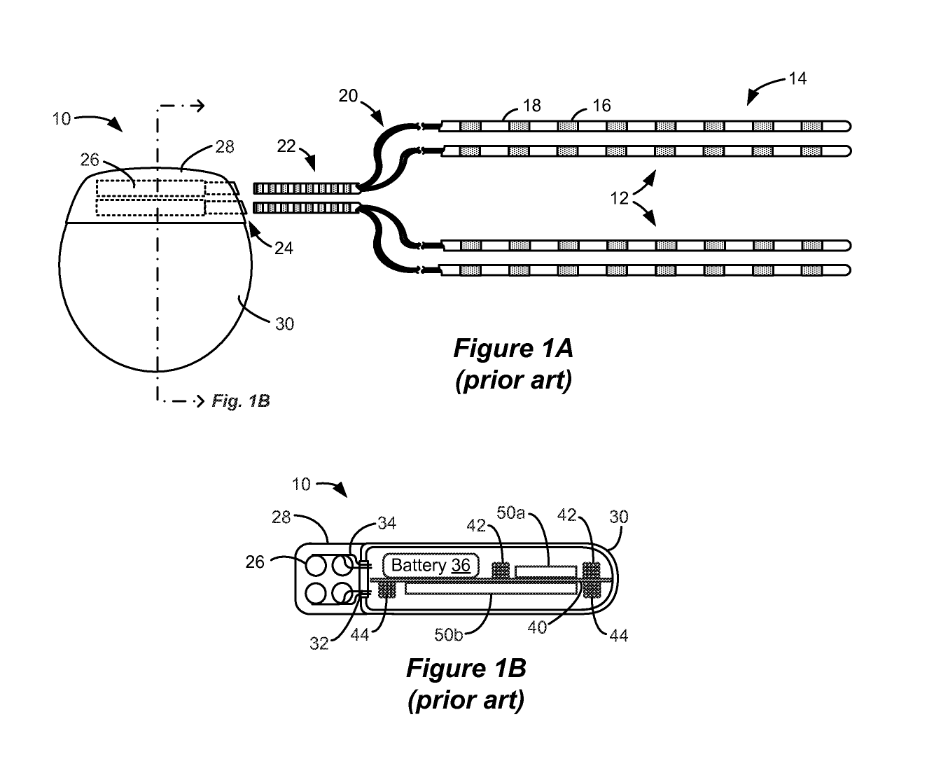

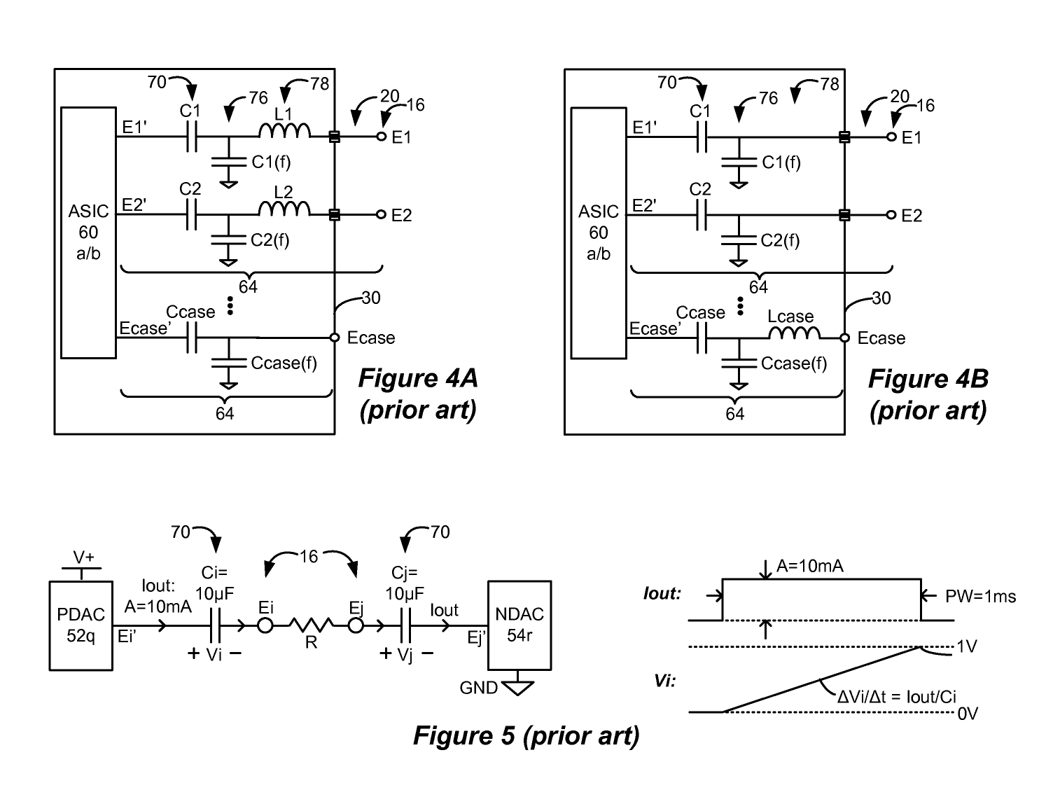

If PCB 140 retains its size, additional space freed on PCB 140 by virtue of the smaller DC-blocking capacitors 170 can be used to accommodate additional components 190 as might be beneficial to expanding IPG functionality. Such other components 190 could comprise circuitry moved from elsewhere in the IPG 10, such as other circuitry 50a or 50b normally present on the top or bottom surfaces of the PCB (FIG. 1B), the microcontroller 62, either or both of ASICs 60a and 60b, etc. In another example, and although not shown, additional components 190 can comprise the EMI/MRI filter capacitors 76 and/or the filtering inductors 78 discussed earlier with reference to the 2014/0155970 Publication, and as shown in FIGS. 4A and 4B. Additional components 190 can also include additional DC-blocking capacitors 170, thus allowing the number of electrodes 16 the IPG 10 can support to be increased (e.g., from 32 to 64).

Even further space savings on the IPG PCB can be had by embedding the DC-blocking capacitors 170 in the PCB, as discussed in U.S. Patent Application Publication 2015/0157861. As the '861 Publication teaches, DC-blocking capacitors can be embedded in different manners, two of which are shown in FIGS. 11B and 11C.

As shown in the cross-section of FIG. 11B, the DC-blocking capacitors 170 are wholly recessed below the top and bottom surfaces of the PCB 140′ where components (e.g., 60a/b, 62, 190) are traditionally coupled to PCB conductive layers 182 at contacts 184 formed in top and bottom dielectric solder mask layers of the PCB 140′. The solderable terminals 172 of embedded DC-blocking capacitors 170 are electrically coupled to one of the conductive layers 182 between various dielectric layers (prepreg; FR4, etc.) in the PCB 140′. PCB vias 186 can be used if necessary to route signals from one conductive layer 182 to another. As a result of recessing the DC-blocking capacitors 170 into the PCB 140′, components 190 can occupy top and bottom surfaces of the PCB 140′ above and below area 180′ where the capacitors 170 are embedded. In short, the embedded DC-blocking capacitors 170 take up none of the top or bottom surfaces areas of the PCB 140′, allowing the PCB 140′ to be made that much smaller, or again allowing the PCB 140′ to include additional components.

As shown in the cross-section of FIG. 11C, the DC-blocking capacitors 170 are again embedded in the PCB 140″ but are not formed using discrete components, such as those containing ceramic packages. Instead, the DC-blocking capacitors 170 are formed by using and patterning the conductive layers 182 surrounding a thin high-quality dielectric layer 188 built into the PCB 140″ itself. PCB suppliers offer materials for this purpose, such as ceramic-filled epoxies sandwiched between layers of copper foil. See, e.g., “3M Embedded Capacitance Material (ECM),” 3M Company (2013). Presently, the capacitances of such materials are rated at 3.1 nF/cm². At this capacity, and assuming a 10 cm² PCB 140″, 33 DC-blocking capacitors 170 can be formed each having a capacitance of about 0.9 nF. The improved circuitry can operate at such capacitances values, although the number of transitions (N) would increase while the time between transitions (tₜ) would decrease.

The improved circuitry can also be used in conjunction with the “DC-blocking capacitor minimization” prior art techniques discussed earlier (U.S. Pat. No. 7,881,803; US Pub. 2010/0268309 and 2005/0245970) which reduce the number of DC-blocking capacitors relative to the number of supported IPG electrodes, thus promoting even further space savings. As noted earlier, these techniques can give rise to additional design complexities, although such complexities may not be prohibitive. Use of the improved circuitry in conjunction with such prior art techniques can also be used in conjunction with the disclosed techniques for embedding DC-blocking capacitors in the PCB 140, as just discussed with respect to FIGS. 11B and 11C.

To this point, the case electrode 30 (Ecase) has been treated more or less synonymously with the other lead electrodes 16 (Ei), in that it may be selectable as an anode or cathode, and can contain the same DC-blocking capacitor 170 in its electrode current path 64, etc. However, the case electrode 30 may in some IPG architectures be governed by different considerations. For example, it may merely couple to a passive reference voltage (such as ground) as opposed to being driven with a current; it may or may not have a DC-blocking capacitor; it may or may not contain filtering components, or may contain such components but with different values, etc. Thus, while the case electrode may comprise one of the plurality of electrodes Ei as recited in the claims below, the case electrode may also be excluded from that set.

A “source circuit” as used in the claims below should be understood as a constant or variable current source or sink, or a constant of variable voltage source, or combinations thereof.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. An implantable stimulator device, comprising:
an electrode configured to contact tissue of a patient;
a source circuit configured to provide a biphasic current pulse to an output, wherein the biphasic current pulse comprises a first phase and a second phase;
a capacitor through which the biphasic current pulse is passed from the output to the electrode; and
a control circuit configured to measure a charge passed through the capacitor during the first phase and the second phase.

2. The device of claim 1, further comprising a circuit board for carrying the source circuitry.

3. The device of claim 2, wherein the capacitor is embedded between a top surface and a bottom surface of the printed circuit board.

4. The device of claim 3, wherein the capacitor comprises a packaged component.

5. The device of claim 3, wherein the capacitor comprises a dielectric layer, and wherein the dielectric layer comprises a layer of the circuit board.

6. The device of claim 1, wherein the source circuit and the output are integrated within an integrated circuit.

7. The device of claim 6, further comprising a switching network for passing the biphasic current pulse from the output to the electrode, wherein the switching network comprises a plurality of switches, and wherein the plurality of switches are integrated within the integrated circuit.

8. The device of claim 7, wherein the capacitor is integrated within the integrated circuit.

9. The device of claim 1, further comprising a switching network for passing the biphasic current pulse from the output to the electrode, wherein the control circuit is further configured to control the switching network to alternate a direction of the biphasic current pulse in accordance with a voltage across the capacitor.

10. The device of claim 9, wherein the control circuit is configured to control the switching network so that the voltage across the capacitor varies between an upper threshold and a lower threshold.

11. The device of claim 10, wherein the control circuit is further configured to measure the charge passed through the capacitor during the first phase and the second phase by counting a number of transitions of the direction of the current as it alternates during the first and second phases of current pulse.

12. The device of claim 11, wherein the control circuit is configured to end the second phase of the biphasic current pulse when the number of transitions during the first phase equals the number of transitions during the second phase.

13. The device of claim 12, wherein the control circuit is further configured to end the second phase when the voltage across the capacitor equals zero volts.

14. The device of claim 1, wherein the source circuit comprises a constant current source.

15. The device of claim 1, wherein an amplitude of the current is variable during the first and second phases of the biphasic current pulse.

16. The device of claim 1, further comprising a case for housing the source circuit, the output, the switching network, and the capacitor, wherein the electrode is coupled to the case by lead.

17. The device of claim 1, further comprising a case for housing the source circuit, the output, the switching network, and the capacitor, wherein the electrode is carried by the case.
